(19) United States
(12) Patent Application Publication
(10) Pub. No.: US 2006/0105400 A1
(43) Pub. Date: May 18, 2006

(54) Elisa kits for detecting collagenase 3 as a proenzyme and in an activated form in body fluids and cell culture supernatants

(76) Inventors: Matthias Dettloff, Berlin (DE); Thorsten Stroh, Berlin (DE); Peter Sveshnikov, Moscow (RU); Peter Bendzko, Berlin (DE)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 10/523,077

(22) PCT Filed: Aug. 27, 2003
(86) PCT No.: PCT/DE03/02864

§ 371 Date: Aug. 11, 2005

(30) Foreign Application Priority Data
Aug. 29, 2002 (DE) 202 13 551.9
Mar. 28, 2003 (DE) 103 14 404.8

(51) Int. Cl. G01N 33/53; G01N 33/537

(52) U.S. Cl. 435/7.5; 435/7.92

(57) ABSTRACT

The invention relates to ELISA kits for detecting pro-collagenase 3 and activated collagenase 3 in body fluids, especially in human serum and synovial fluid, and in cell culture supernatants, and monoclonal antibodies which specifically recognise said antigens. Said ELISA kit comprises the following separately packed elements: a) a solid carrier comprising monoclonal antibodies which are bound thereto and sensitively and specifically bind human procollagenase 3 or activated collagenase 3; b) human recombinant procollagenase 3 or activated collagenase 3 as a standard for the quantitative determination of said enzyme; c) a buffer for producing a standard series of the recombinant collagenase 3; d) a buffer for diluting the body fluids to be analysed; e) a conjugate which is marked in such a way that it can be detected and which binds to collagenase 3; and f) a substrate which enables the visualisation of said conjugate which is marked in such a way that it can be detected. The monoclonal antibodies cited in a) are preferably monoclonal antibodies which are formed from the hybridoma with the deposit number DSM ACC 2572. Said ELISA kit can be used in medical fields, especially for medical diagnosis, especially for controlling the development of rheumatoid arthritis, systemic lupus erythematosus and tumour diseases.

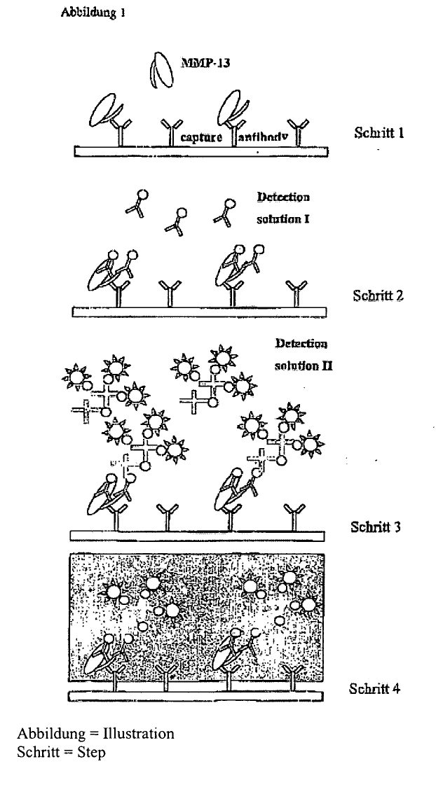

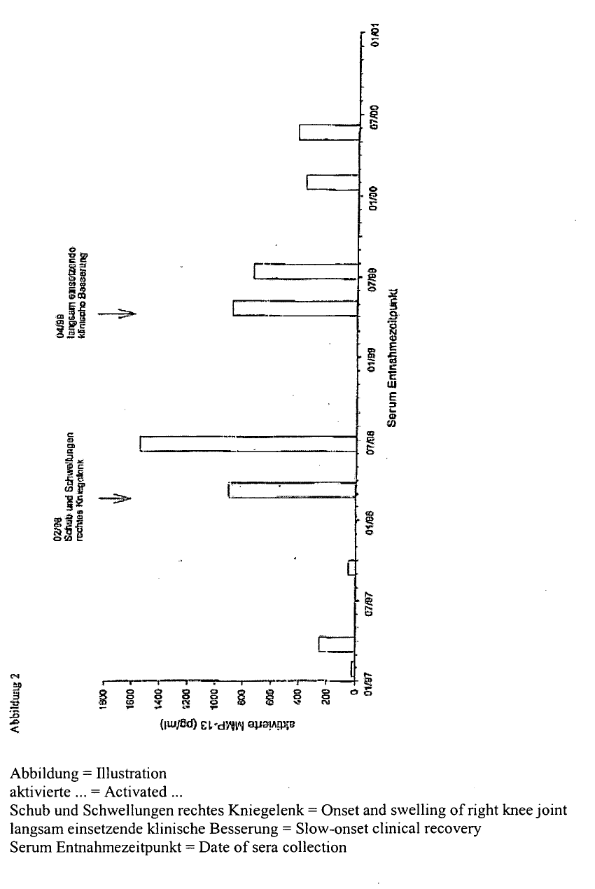

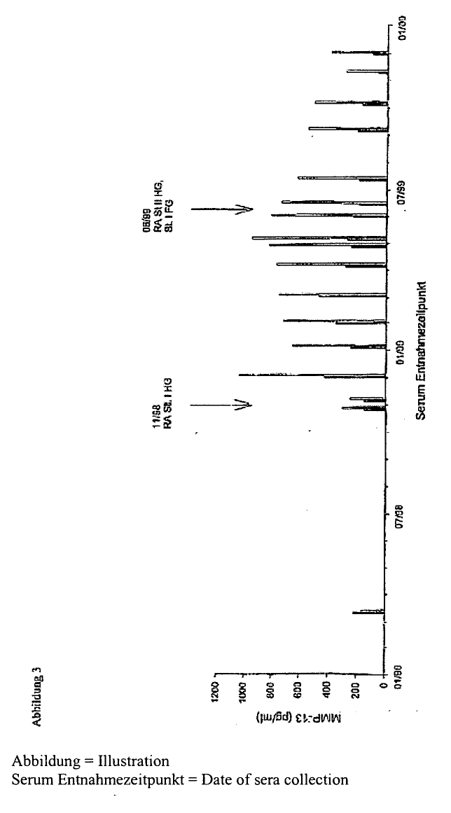

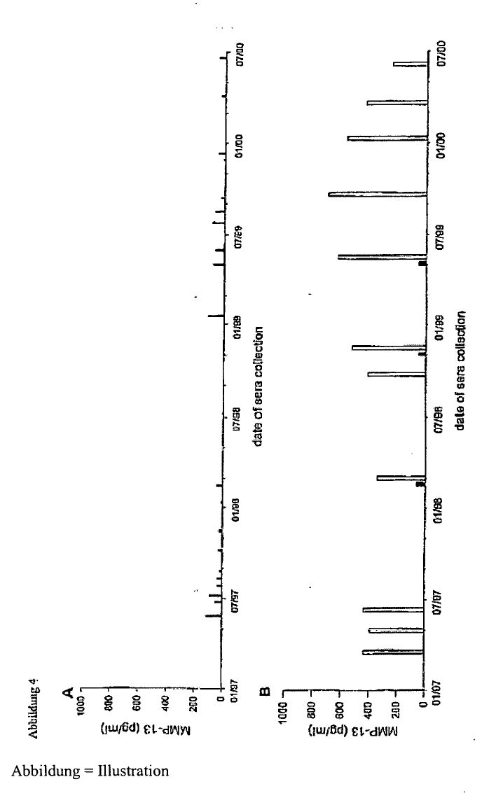

The invention relates to ELISA kits for detecting collagenase 3 as a proenzyme and in activated form in body fluids, especially in human serum and synovial fluid and in cell culture supernatants, and monoclonal antibodies that specifically detect collagenase 3 in latent and activated form.

Areas of application are medicine and in this case especially diagnostics, in particular the progressive course assessment of inflammatory rheumatic diseases (rheumatoid arthritis), systemic lupus erythematosus (SLE) with involvement of organs and tissue proliferation and of tumorous diseases (e.g. mammary and colorectal carcinomas).

The enzyme-linked immunosorbent assay (ELISA) technique is the current technological standard in clinical laboratories. Using this technology, it is possible to determine marker proteins, among other things, for specific diseases in body fluids of patients.

Matrix metalloproteinases (MMPs) form a family of secreted and membrane-bound endoproteinases that hydrolyze the extracellular matrix proteins (Nagase, H. and Woessner, F., Jr., J. Biol. Chem. 1999, 274, 21491-21494). On the basis of their preferred substrates and structural features, it is possible to classify MMP's into collagenases, gelatinases, stromelysines and membrane-type metalloproteases.

Collagenase 3 (MMP-13) is released from cells as inactive proenzyme (procollagenase 3, pro-MMP-13) and extracellularly converted into the activated form via splitting off a propeptide.

Both procollagenase 3 and activated collagenase 3 are typically undetectable in fully differentiated adult tissue. However, its occurrence is described in connection with a whole series of destructive medical conditions: In the case of developing breast cancers (NIELSEN B S et al., Cancer Res. 2001 61: 7091-7100), rheumatoid arthritis (Westhoff C S et al., Arthritis Rheum. 1999 42: 1517-1527) and osteoarthrosis (Shlopov B V et al. Arthritis Rheum. 1997 40: 2065-2074), the level of procollagenase 3-mRNA is sharply up-regulated in the affected tissue types. These findings make clear that collagenase 3 is a marker protein of great interest for medical diagnostics.

However, so far there exist no studies for the course of any medical condition, not even for rheumatoid arthritis, regarding the actual level of procollagenase 3 or activated collagenase 3 in body fluids, such as serum or synovial fluid, because to date there has been no satisfactory technological solution available on the market that would have permitted such measurements.

Currently, two products are offered with which the amount of procollagenase 3 may be determined in principle. However, neither test distinguishes between the proenzyme and the activated collagenase 3.

1. Biotrak® Matrix Metalloproteinase-13 ELISA System

The first test, the Biotrak Matrix Metalloproteinase-13 ELISA system, is validated for the body fluids serum and plasma. The problem of the test is the very low sensitivity. Moreover, the test is not validated for analyses of synovial fluid and therefore cannot be used for this purpose.

2. Quantikine® pro-MMP-13 Immunoassay

The second test system on the market is specified exclusively for use in cell cultures and therefore cannot be employed for analyses of procollagenase 3 in body fluids.

Consequently, the object of the invention was to provide an ELISA kit that, in contrast to the tests found on the market, distinguishes itself by high sensitivity and is suitable for detecting procollagenase 3 and the activated form of this enzyme both in body fluids, especially in human serum and synovial fluid, and also in cell culture supernatants. Moreover, the invention should also offer for the first time the opportunity of determining with a high degree of specificity the quantitative relationship between the latent and active forms of this enzyme in cell culture supernatants and body fluids.

The invention is realized according to the claims.

The ELISA kits according to the invention for detecting procollagenase 3 and activated collagenase 3 comprise at least the following separately packed elements:
a) a solid carrier having monoclonal antibodies that are bound thereto and specifically and sensitively bind human procollagenase 3 or activated collagenase 3;
b) human recombinant procollagenase 3 or activated collagenase 3 as a standard for the quantitative determination of this enzyme in body fluids and cell culture supernatants;
c) a buffer for producing a standard series of the recombinant collagenase 3;
d) a buffer for diluting the samples to be analyzed;
e) a detectably marked conjugate that binds to collagenase 3; and
f) a substrate that allows the visualization of the detectably marked conjugate,
   where the antibodies mentioned under a) are preferably either Anti-MMP-13 clone M34 (mouse), and especially preferably monoclonal antibodies that are formed from the hybridoma having the deposit number DSM ACC 2572, or preferably Anti-MMP-13 clone EE1 (mouse).

A combination of two components, the first being biotinylated antibodies that bind to collagenase 3 and the second being a high-polymer streptavidin conjugate that binds to the biotinylated antibodies, may be used for the detectably marked conjugate.

Alternatively, conjugated antibodies that bind to collagenase 3 may also be used for this purpose.

The antibodies that function as conjugate may be monoclonal and/or polyclonal antibodies.

Human recombinant procollagenase 3, which was expressed in eukaryotic cells (Sf9 cells), is used as the standard for quantitative determination of the procollagenase 3 in body fluids and cell culture supernatants. It is available either in solution or in freeze-dried form, wherein it is preservable for several months without a loss of quality. If in freeze-dried form, the recombinant procollagenase 3, before being used, must first be reconstituted by adding distilled water. Human recombinant activated collagenase 3 is produced from the aforementioned procollagenase 3 by adding acetamino-phenyl-mercury-acetate (APMA) and used in the same manner.

The buffer provided for diluting samples to be analyzed contains sodium citrate in addition to blocking and stabilizing substances. Surprisingly, it was shown that this reagent for the preparation of human serum is especially suitable for the measurement of collagenase 3.

The buffer for producing a standard series of recombinant procollagenase 3 or the activated collagenase 3 for the measurement of this marker in serum contains human serum.

Microtiter plates to which the monoclonal antibodies according to the invention are bound are preferably used as solid carriers. These microtiter plates are produced in such a manner that they can be stored for several months without a loss of quality.

The subject matter of the invention also includes monoclonal antibodies that specifically detect and bind collagenase 3 as a proenzyme or activated enzyme, these monoclonal antibodies being produced from hybridoma cell lines having the deposit number DSM ACC 2572 or having properties like the monoclonal antibodies from the hybridoma cell line having the deposit number DSM ACC 2572.

Also pertaining to the invention are antibodies that have properties like the monoclonal antibodies from the hybridoma cell lines with the deposit number DSM ACC 257 that, however, may be altered biochemically or molecular biologically or may be synthetic, where the antibodies or parts that are not necessary for detecting the procollagenase 3 are completely or partially lacking or these parts are replaced by others.

The ELISA kits according to the invention enable the detection of procollagenase 3 and of activated collagenase 3 in body fluids, especially in human serum and synovial fluid, as well as in cell culture supernatants having a high sensitivity, and therefore make these potential disease markers more accessible to medical diagnostics.

In comparison to the Biotrak® Matrix Metalloproteinase-13 ELISA system, the sensitivity of the ELISA kits of the invention is higher by a factor of ten. Expressed in numbers, the lower detection limit of the ELISA is 4 pg procollagenase 3 or 6 pg activated collagenase 3 per ml of specimen.

The standard curve determined in the measurement by analysis of a human recombinant procollagenase 3 carried along or the activated collagenase 3 allows a rapid calculation of the collagenase level in samples using the regression function based on the standard curve. A further critical advantage is that the ELISA kits may be stored in the refrigerator, which substantially improves their convenience and user-friendliness.

The ELISA kits according to the invention for detecting procollagenase 3 or activated collagenase 3 may be kept by the consumer for at least one month overall. The production is carried out in accordance with EN 46001 and EN ISO 9001 standards. The ELISA kits for the first time offer the possibility of analyzing synovial fluid.

In a study using patient sera, it is shown for the first time that, using the ELISA kits according to the invention, collagenase 3 is a marker for monitoring the progressive course of rheumatoid arthritis, but also of severe cases with organ involvement and tissue proliferation of systemic lupus erythematosus. An increase of the content of MMP-13 in the serum detected using the ELISA kits according to the invention precedes an acute clinical deterioration of the medical condition in the case of severe progressive forms of rheumatoid arthritis. The enzyme is not detectable at any time in the serum; therefore, this marker is primarily suited for the progressive prognosis of selected diseases, especially for a preventative start of treatment before symptoms become clinically noticeable in the patient.

The subject matter of the invention is also the use of collagenase 3
as a serological marker for the diagnosis and especially for the progressive course assessment of inflammatory rheumatic diseases, especially rheumatoid arthritis and
as a serological marker for diagnosis and especially for the progressive course assessment of systemic lupus erythematosus, in particular for the progressive course prognosis when there is simultaneous tissue proliferation (tumor formation).

Collagenase 3 may also be used as a serological marker for diagnosis and for progressive course assessment of other tumorous diseases, especially mammary and colorectal carcinomas.

Collagenase 3 may also be employed as a serological marker for the diagnosis and progressive course assessment of other diseases in which an increase of collagenase 3 is described.

The invention is explained in detail below using examples and illustrations.

### EXAMPLE 1

### Production and Screening of the Monoclonal Antibodies

Mice were used for the immunization. Human recombinant procollagenase 3 that was expressed in SF9 cells functioned as antigen. The antigen was prepared in the following manner: 50 μg of MMP-13 in 100 μl of PBS+100 μl of 6M urea were prepared. Then 100 μl of CFA or IFA were added to this solution.

The injection was carried out according to the following scheme:

Day 0: 50 μg of MMP-13 intraperitoneal in CFA

Day 13: 50 μg intraperitoneal in IFA

Day 41: 50 μg intravenous in 1 ml of PBS

Day 44: Hybridization of pre-lymphocytes with SP 2/0 myeloma and spleen cells.

Three hybridizations from one and the same spleen were carried out using different lymphocyte quantities. The third hybridization was successful.

Supernatants of the mature hybridomas were tested in the ELISA. For this purpose 100 μl of recombinant human procollagenase 3 (1 μg/ml in PBS) were immobilized in the wells of a titer plate overnight at 4° C. and after 3 washing steps (PBS with 0.05 % Tween® 20) blocked for 2 hours using a blocking buffer (1% BSA in PBS). At that point 50 μl of the cell culture supernatants, as well as positive and negative controls, were added into the wells for one hour (37° C.). After this incubation, the plate was washed three times, and for another hour (37° C.) 100 μl of anti-mouse IgG (H+L)-POD-conjugate were added. After another five washings, the POD content was detected in the wells using 100 μl of TMB substrate (20 min, RT) for each one. The reaction was stopped using 50 μl 2 M H₂SO₄ and the absorption measured at 450 nm.

The positive hybridoma supernatants in the ELISA were cloned and recloned until attaining monoclonality. 5 independent monoclonal antibodies were obtained, from which a total of 12 subclones having partially altered affinities were extracted. A hybridoma cell line that produces the monoclonal antibody anti-MMP-13 clone M34 (mouse) according to the invention (IgGl), was deposited at the Deutschen Sammlung von Mikroorganismen und Zellkulturen GmbH [German Microorganism and Cell Culture Collection GmbH] (DSM ACC 2572Z) in Braunschweig under the number DSM ACC 2572 on Aug. 27, 2002.

### EXAMPLE 2

### Performing the ELISA's

The principle of the ELISA's is depicted in Illustration 1. To perform the ELISA's, a dilution series is developed from the human recombinant collagenase 3 as a standard, which contains the recombinant procollagenase 3 (or activated collagenase 3) in the following concentrations: 1000 pg/ml, 500 pg/ml, 250 pg/ml, 125 pg/ml, 63 pg/ml, 32 pg/ml, 16 pg/ml, 0 pg/ml (Start of the dilution series for the determination of activated collagenase 3: 2000 pg/ml). If the collagenase 3 content in serum is to be determined, the standard dilution is produced using a special buffer system that contains 10% human serum. At this point 100 μl of these standard dilutions are pipetted, in repeat determination, into the microwells of the microtiter plate to which monoclonal antibodies from the hybridoma cell line having the deposit number DSM ACC 2572 are bound. The samples to be measured (cell culture medium, synovial fluid or serum) are diluted with the buffer provided for the sample preparation. From the diluted samples, 100 μl are then likewise applied at this point in repeat determination.

After 120 minutes of incubation on a shaker at room temperature, the microtiter plate is washed four times using the washing buffer, and thereafter the remaining liquid is removed by blotting on paper towels. Next comes the addition of 100 μl of detection solution 1, which contains biotinylated antibodies, in all wells of the microtiter plate that are used. These are either polyclonal antibodies or monoclonal antibodies or a cocktail made of a plurality of monoclonal antibodies. After 90 minutes of incubation, the microtiter plate is again washed four times and blotted on a paper towel. Detection solution 2, comprising a streptavidin peroxidase conjugate and a dilution buffer is produced according to instructions and again 100 μl are pipetted into the corresponding microwells of the microtiter plate. A further incubation of 30 minutes follows. Thereafter, the microtiter plate is washed five times and blotted dry, is provided with 100 μl of substrate solution (tetramethyl benzidine) per microwell and incubated in the dark for 15 minutes. At the end of this time, 100 μl of stopping solution (0.5 M sulfuric acid) are added to the microwells and the microtiter plate is measured at 450 nm in a microtiter plate reader. The magnitude of the optical absorption corresponds to the collagenase 3 content.

### EXAMPLE 3

### Analysis of Body Fluids Using the ELISA Kit

A larger number of patient sera having different disease aspects was tested using the ELISA kits.

TABLE 1
Measurement of pro-MMP-13 and activated MMP-13 using
the two InviLISA MMP-13 kits. The table indicates
the sample size collected and the number of positive
specimen (in each test and in aggregate).

| Disease | Collection(s) | Pro MMP-13 | Act MMP-13 | total pos. |
|---|---|---|---|---|
| RA | 145 | 7 | 22 | 20% |
| PSS | 50 | 0 | 0 | 0% |
| SLE | 40 | 0 | 1 | 3% |
| Myositis | 33 | 0 | 1 | 3% |
| Schlerodermia | 15 | 0 | 0 | 0% |
| Vasculitis | 21 | 0 | 0 | 0% |
| Fibromyalgia | 15 | 0 | 0 | 0% |
| Blood donor | 160 | 3 | 1 | 2% |

RA = rheumatoid arthritis;
PSS = primary Sjogren's Syndrome;
SLE = systemic lupus erythematosus.

Table 1 shows the results of these measurements: While in a control population from blood donors only about 2% reacted positive in the tests, 20% of the sera from patients with rheumatoid arthritis showed positive signals.

Sera from patients having other diseases (systemic lupus erythematosus, myositis, schlerodermia, vasculitis, fibromyalgia) were comparable to the control population. Moreover, serum samples of patients with rheumatoid arthritis were measured that had been collected over the course of two to three years. Two curves are shown as examples (Illustrations 2 and 3). Illustration 2 clearly shows increased active MMP-13 values immediately before the onset of clinical deterioration, which is accompanied by a massive swelling of the right knee joint. The slow-onset clinical recovery follows a drop in the MMP-13 titer.

Illustration 3, like illustration 2, shows the particular suitability of the activated collagenase 3 as a progression marker for rheumatoid arthritis. In the first six months of the analysis time frame, the measured MMP-13 values did not increase or fall below the defined cutoff of 300 pg/ml of MMP-13. The patient involved was X-rayed in November 1998; the wrist joint being classified as 1^{st} degree according to the Larsen scale. In December 1998, the patient suffered a massive onset that went along with measured sharply increased MMP-13 values. In June 1999 control X-rays showed a progressive destruction of the wrist joint (Larsen 2^{nd} degree) and an incipient destruction of the ankle joint (Larsen 1^{st} degree).

As a control, consecutive measurements were performed on sera from patients with Sjögren's Syndrome.

At no time did these show increased MMP-13 values. (Data not shown).

Depicted in Illustration 4 are two exemplary progressive measurements of patients with systemic lupus erythematosus (SLE). While the sera from patients with severe disease progression themselves exhibit no increased MMP-13 values, or only marginal ones (Illustration 4 A, SLE with renal participation, very severe symptomology), sera from SLE patients with tissue proliferation showed increased values of activated MMP-13 (Illustration 4 B, SLE with renal participation, membrane-proliferating type IVa glomerulonephritis, a severe symptomology). These measurements indicate that MMP-13 can be an indicator for tumor growth.

Illustrations Legend

Illustration 1: Detection Method Concept

Step 1: Incubation of standards or samples on the titer plate. Specific binding of collagenase 3 (MMP-13) as a proenzyme or in activated form (duration: 120 minutes)

Step 2: Detection of the bound collagenase 3 (MMP-13) with biotinylated antibodies (duration: 90 minutes)

Step 3: Addition of streptavidin peroxidase conjugate (duration: 30 minutes)

Step 4: Color development after addition of TMB substrate (duration: 15 minutes)

Illustration 2: Measurement of Activated MMP-13 in the Serum of a Patient with Rheumatoid Arthritis (Larsen III, DAS Score>3.8).

No increased Pro-MMP-13 values were detectable in the samples. The increase of the MMP-13 values in the serum right at the time of an onset and the subsequent dropping of the values during the remission (arrows) are clear.

Illustration 3: Measurement of Pro-MMP-13 (Black Bars) and Activated MMP-13 (Gray Bars) in the Serum of a Patient with Rheumatoid Arthritis.

Explanations in the text. RA=rheumatoid arthritis; St.=stage; HG=wrist, FG=ankle.

Illustration 4: Measurement of Pro-MMP-13 (Black Bars) and Activated MMP-13 (Gray Bars) in Sera from Two Patients with SLE.

A: SLE patient with renal participation, nephrotic syndrome, very severe symptomology. B: SLE-patient with renal participation, severe symptomology, proliferating type IVa glomerulonephritis. Explanations in the text.

1. ELISA kits for detecting procollagenase 3 and activated collagenase 3 in body fluids, especially in human serum and synovial fluid, and in cell culture supernatants, comprising at least the following separately packed elements:
a) a solid carrier having monoclonal antibodies which are bound thereto and sensitively and specifically bind human procollagenase 3 or activated collagenase 3;
b) human recombinant procollagenase 3 or activated collagenase 3 as a standard for the quantitative determination of this enzyme in body fluids;
c) a buffer for producing a standard series of the recombinant procollagenase 3 or activated collagenase 3;
d) a buffer for diluting the samples to be analyzed;
e) a detectably marked conjugate that binds to collagenase 3;
f) and a substrate that allows the visualization of the detectably marked conjugate.

2. The ELISA kit as described in claim 1, wherein the monoclonal antibodies that are bound to the solid carrier are preferably monoclonal antibodies that are formed of the hybridoma having the deposit number DSM ACC 2572.

3. An ELISA kit as described in claim 1 wherein a conjugated antibody is used as a detectible labeled conjugate, said conjugated antibody having a binding affinity to procollagenase 3 or to activated collagenase 3.

4. An ELISA kit as described in claim 2, wherein a conjugated antibody 3 is used as a detectible labeled conjugate, and said conjugated antibody has binding affinity to procollagenase 3 or to activated collagenase 3.

5. The ELISA kit as described in claim 3 wherein the antibodies that function as the conjugate are monoclonal and/or polyclonal antibodies.

6. The ELISA kit as described in claim 1, wherein the substances used as conjugates may be conjugated with all standard substances, preferably with:
horseradish peroxidase
alkaline phosphatase.

7. The ELISA kit as described in claim 1, wherein the human recombinant collagenase 3 used as the standard was expressed in eukaryotic cells and is present in solution or lyophilizated.

8. The ELISA kit as described in claim 1, wherein the buffer for diluting the body fluids and cell culture supernatants to be analyzed contains sodium citrate.

9. The ELISA kit as described in claim 1, wherein microtiter plates or standard protein chip technologies are used as solid carriers.

10. Monoclonal antibodies that specifically detect and bind procollagenase 3, these monoclonal antibodies having properties like the monoclonal antibodies from the hybridoma cell line having deposit number DSM ACC 2572.

11. The monoclonal antibodies as described in claim 10, whereby the monoclonal antibodies can be changed in a biochemical or molecular biological manner or synthetically, whereby the antibodies or parts that are unnecessary for the detection of procollagenase 3 are lacking, wholly or in part, or these parts are replaced by others.

12. The monoclonal antibodies as described in claim 10 that are produced from the hybridoma cell line having the deposit number DSM ACC 2572.

13. Hybridoma cell line having the deposit number DSM ACC 2572

14. Monoclonal antibodies that detect and bind activated collagenase 3 in a specific and sensitive manner, these antibodies having no affinity for procollagenase.

15. The monoclonal antibodies as described in claim 10, whereby the monoclonal antibodies can be changed in a biochemical or molecular biological manner or synthetically, whereby the antibodies or parts that are unnecessary for the detection of activated collagenase 3 are lacking, wholly or in part, or these parts are replaced by others.

16. A method for using collagenase as a serological marker for diagnostics and especially for monitoring the course of diseases.

17. The method of claim 16, wherein said disease is systemic lupus erythematosus, especially for the developmental prognosis when there is tissue proliferation (tumor formation).

18. The method of claim 16, wherein said disease is tumorous disease, especially mammary carcinomas and colorectal carcinomas.

19. The method of claim 16, wherein said disease is selected from diseases in which an increase of collagenase 3 is described in the scientific literature.

20. An ELISA kit according to claim 3, wherein said conjugated antibody is conjugated with biotin and the detectible labeled conjugate further comprises streptavidin-conjugate, which binds to biotinylated antibodies.

21. The method of claim 16, wherein said disease is selected from inflammatory rheumatic diseases, especially rheumatoid arthritis.
